(19) United States
(12) Patent Application Publication
(10) Pub. No.: US 2012/0135526 A1
(43) Pub. Date: May 31, 2012

(54) LOW-PRESSURE BIOLISTIC BARRELS

(76) Inventor: Kenneth Greenberg, Oakland, CA (US)

(21) Appl. No.: 13/300,288

(22) Filed: Nov. 18, 2011

Related U.S. Application Data
(60) Provisional application No. 61/416,682, filed on Nov. 23, 2010.
(60) Provisional application No. 61/503,883, filed on Jul. 1, 2011.

(51) Int. Cl. A61M 37/00; C12N 15/87; C12M 1/00

(52) U.S. Cl. 435/459; 435/285.3; 604/140

(57) ABSTRACT

Low pressure biolistic barrels and biolistic devices including the same are provided. Aspects of the biolistic barrels include the presence of one or more pressure-reducing elements. Also provided are kits which include the biolistic barrels, as well as methods of delivering a molecule to a target site with the biolistic barrels and devices that include the same. The devices and methods described herein find use in a variety of applications, including in vivo and in vitro high-precision delivery applications.

### REFERENCE TO RELATED APPLICATION

Pursuant to 35 U.S.C. §119(e), this application claims priority to U.S. Provisional Patent Application No. 61/416,682 filed on Nov. 23, 2010 and to U.S. Provisional Patent Application No. 61/503,883 filed Jul. 1, 2011; the disclosures of which applications are herein incorporated by reference in their entirety.

### REFERENCE TO GOVERNMENT SUPPORT

This invention was made in part with government support under Grant No. 32 EY018790-01 awarded by the National Institutes of Health. The government has certain rights in this invention.

### INTRODUCTION

Recent advancements in molecular biology have provided a number of techniques for manipulating the genetic material of a cell or organism. A particular subset of genetic engineering technology has facilitated the introduction of foreign genes in cells to alter their biological characteristics and morphology. The introduction of foreign genes in host cells within plants, for example, has been shown to improve vital traits such as insect resistance, frost resistance, and nutrient compositions. In recent years, the introduction of foreign genetic material in the form of gene therapy has been applied in treating diseased human tissue both in vivo and in vitro.

The introduction of foreign genetic material into a cell is commonly referred to as gene “transformation” in bacterial cells and gene “transfection” in animal cells. Both gene transformation and gene transfection can be conducted using several different approaches. One approach utilizes bacterial plasmids or viral vectors as carriers for delivering genes into cells. Other approaches, such as electroporation and microinjection, involve the physical disruption of a cell membrane to allow the introduction of foreign genetic material. Electroporation involves the use of electrical impulses to increase cell membrane and cell wall permeability to DNA contained in a solution surrounding the cell. Microinjection is a technique involving the injection of DNA directly into a cell nucleus using an ultrafine needle. Lipofection, also known as liposome transfection, is a technique used to introduce foreign genetic material into a cell by means of liposomes, which are vesicles which possess phospholipid bilayers that can merge with a cell membrane.

Biolistic transfection is a physical method of gene transfection in which high density, sub-cellular sized particles coated with foreign genetic material are accelerated to high velocity to carry the genetic material into cells. Because biolistic transfection does not depend on specific receptors or biochemical features typically present on cell surfaces, it can be readily applied to a variety of biological systems including plants and mammalian tissue. Also, since biolistic transfection involves the delivery of particles to cells at high velocity, it can overcome physical barriers to effective gene transfer, such as the stratum corneum of the epidermis, inner limiting membrane of the retina, and the cell wall of plants, for example.

### SUMMARY

Low pressure biolistic barrels and biolistic devices including the same are provided. Aspects of the biolistic barrels include the presence of one or more pressure reducing elements. Also provided are kits which include the biolistic barrels, as well as methods of delivering a molecule to a target site with the biolistic barrels and devices that include the same. The devices and methods described herein find use in a variety of applications, including in vivo and in vitro high-precision delivery applications (where such applications may be characterized by substantially little, if any, tissue damage).

### BRIEF DESCRIPTION OF THE FIGURES

FIG. 1 depicts a biolistic barrel, according to embodiments of the present disclosure.

FIG. 2 depicts a biolistic device that includes a biolistic barrel, according to embodiments of the present disclosure.

FIG. 3 depicts a longitudinal cross-section of a biolistic device, according to embodiments of the present disclosure.

FIG. 4 depicts a magnified view of rabbit retinal neurons subsequent to in vivo delivery of biolistic particles using a biolistic device, according to embodiments of the present disclosure.

FIG. 5 depicts a biolistic barrel in which the barrel includes an integrated particle cartridge/handle and a flexible tubing with an adapter, according to embodiments of the present disclosure.

FIG. 6 depicts a biolistic device, according to embodiments of the present disclosure. FIG. 6A shows a photograph of a biolistic barrel and an actuator. FIG. 6B shows a photograph of an enlargement of the biolistic barrel of FIG. 6A. FIG. 6C shows a photograph of the internals of the actuator of FIG. 6A. FIG. 6D shows a schematic of the solenoid trigger circuit of the actuator of FIG. 6A.

FIG. 7 depicts a magnified view of rabbit retinal neurons subsequent to in vivo delivery of biolistic particles using a biolistic device, according to embodiments of the present disclosure. FIGS. 7A and 7B show in vivo fundus fluorescent and bright field images, respectively, of rabbit retina two weeks following in vivo transfection with the biolistic device. FIGS. 7C and 7D show images of tdTomato expression shown ex vivo under low magnification in FIG. 7C and in high magnification in FIG. 7D. Scale bars are 500 μm in FIG. 7C and 50 μm in FIG. 7D.

FIG. 8 depicts fluorescent images of retinal neurons transfected with a biolistic device, according to embodiments of the present disclosure. FIGS. 8A, 8B and 8C show fluorescent images of primate retina (viewed en face) transfected with eGFP at different densities; high density transfection (FIG. 8A), medium density transfection (FIG. 8B), and low density transfection (FIG. 8C). FIG. 8D shows a fluorescent image of a transverse section of eGFP expression driven by CAG promoter in neurons and radial Muller glia. FIG. 8E shows a fluorescent image depicting cell specific hCx36 promoter driven eGFP expression in ganglion cells. FIG. 8F shows a fluorescent image of apoptotic cells stained with Yo-Pro following 72 hour culture of primate retina explant. FIG. 8G (en face view) and FIG. 8H (transverse section) show fluorescent images of multiplex transfection of two plasmids with different promoters and transgenes (Cx36-eGFP and HCN4-tdTomato), which simultaneously labeled two different sets of rabbit ganglion cells.

### DETAILED DESCRIPTION

Low pressure biolistic barrels and biolistic devices including the same are provided. Aspects of the biolistic barrels include the presence of one or more pressure reducing elements. Also provided are kits which include the biolistic barrels, as well as methods of delivering a molecule to a target site with the biolistic barrels and devices that include the same. The devices and methods described herein find use in a variety of applications, including in vivo and in vitro high-precision delivery applications.

Before the present invention is described in greater detail, it is to be understood that aspects of the present disclosure are not limited to the particular embodiments described, and as such may, of course, vary. It is also to be understood that the terminology used herein is for the purpose of describing particular embodiments only, and is not intended to be limiting, since the scope of embodiments of the present disclosure will be defined only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within embodiments of the present disclosure, subject to any specifically excluded limit in the stated range. Where the stated range includes one or both of the limits, ranges excluding either or both of those included limits are also included in embodiments of the present disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of embodiments of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that embodiments of the present disclosure are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the present disclosure, aspects of embodiments of the biolistic barrels are described first in greater detail. Next, embodiments of the biolistic devices which include the biolistic barrels are reviewed in greater detail. Following this description, methods of delivering a molecule to a target site are reviewed in greater detail, followed by a description of embodiments for kits that include at least the biolistic barrels (where the kits may further include one or more additional components, such as system components up to and including an entire biolistic delivery system). Finally, a review of the various applications in which the biolistic barrels, biolistic devices, methods, and kits may find use is provided.

### Biolistic Barrels

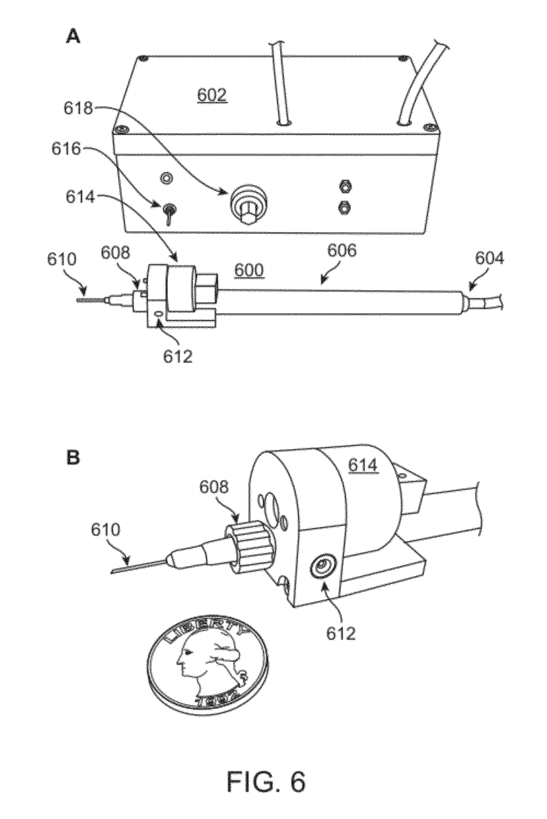

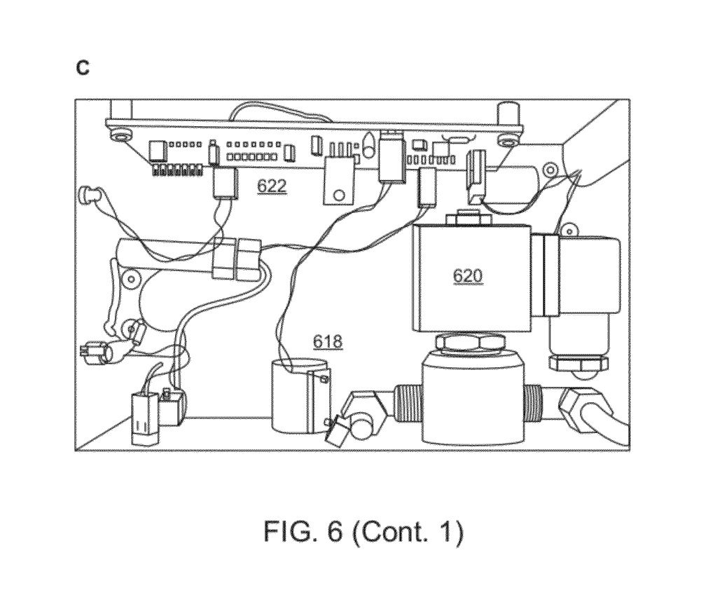

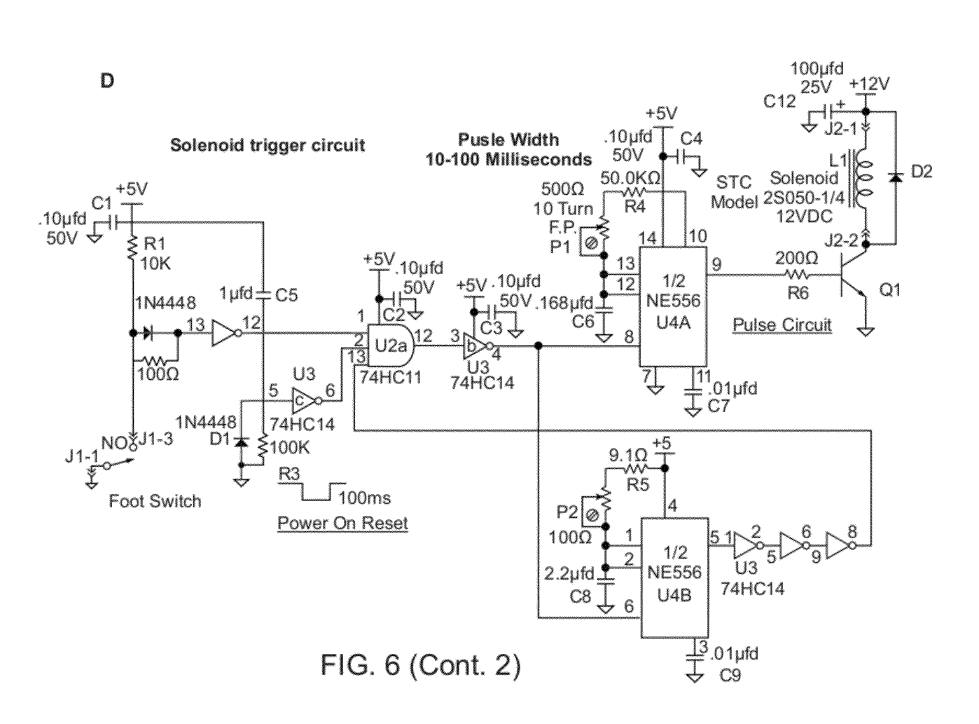

As summarized above, aspects of the invention include biolistic barrels. Biolistic barrels are conduits which allow the delivery of gas and biolistic particles to a target site under gas pressure. In certain embodiments, biolistic barrels are devices configured to operatively connect to a pressurized gas source, such as a pressurized gas source delivered through a biolistic delivery device, e.g., a “gene gun”, a solenoid trigger device (e.g., as described in more detail below and in FIG. 6A), and the like. Biolistic barrels are further configured to receive high pressure gas, e.g., which may or may not include biolistic particles, at a proximal (first) end and convey the gas/particles a distance from the device where the particles then emerge from the barrel at a distal (second) end. In some instances, e.g., as described below, the particles may be present in the barrel and combine with the gas in the barrel, so that the gas/particles emerge from the distal end.

Biolistic barrels of the present disclosure are low pressure biolistic barrels. By “low-pressure” is meant that the gas/biolistic particle mixture that emerges from the biolistic barrel has a low pressure, e.g., as described in greater detail below. Embodiments of the biolistic barrel have the ability to decrease gas pressure within the biolistic barrel by a value of 0.25 psi or more, such as 2.5 psi or more, such as 5 psi or more, such as 10 psi or more, such as 25 psi or more, such as 50 psi or more, including 100 psi or more, between the proximal and distal ends of the barrel. In certain embodiments, during operation, the ratio of pressure exiting at the distal end particle outlet to pressure entering at the proximal end elevated pressure gas inlet of the barrel ranges from 1 to 1000, such as from 1 to 100, for example, from 1 to 20, including from 1 to 10. Although the biolistic barrels are “low-pressure” biolistic barrels, they provide for effective delivery of biolistic particles to target tissue with greatly reduced tissue damage, e.g., as compared to control barrels that are not low pressure biolistic barrels as described herein, e.g., that do not include a pressure reducing element (such as described in greater detail below).

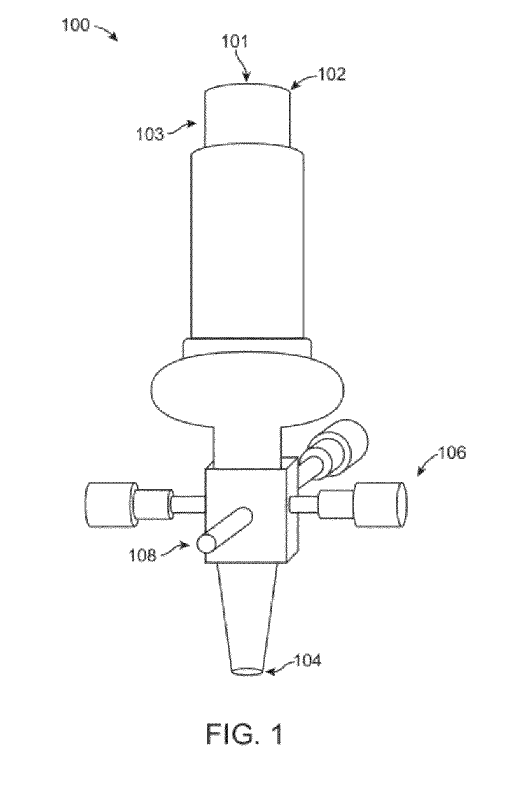

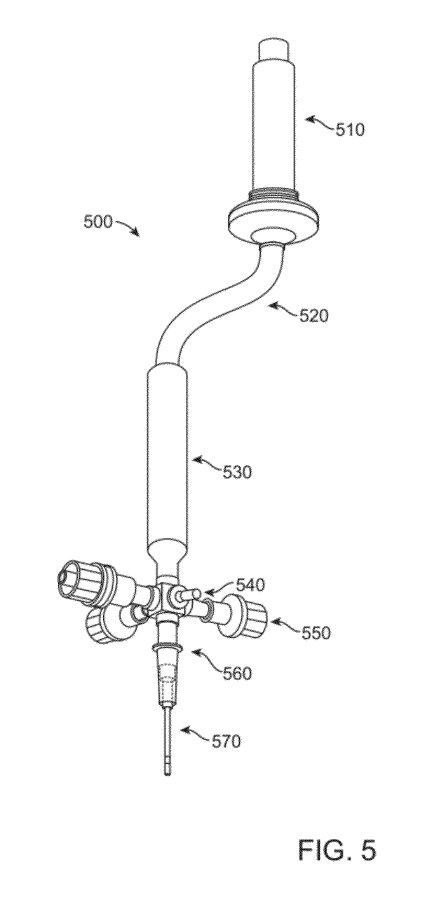

Biolistic barrels according to embodiments of the present disclosure include an elongated body with a defined longitudinal length and a fluid passageway, i.e., a lumen, extending therethrough. As used herein, the term “elongated body” refers to a structure which has a length that is greater than its width, where in some instances the length exceeds the width by 2 fold or more, such as 5 fold or more, including 10 fold or more. The length of the biolistic barrel may vary greatly, e.g., depending on whether the barrel is one that primarily includes just a pressure reducing element, e.g., as shown in FIGS. 1 to 3, or whether the barrel includes one or more additional integrated functionalities, e.g., flexible tubing, integrated handle, particle cartridge, etc., such as depicted in FIG. 5. In some instances, the length of the biolistic barrel ranges from 2 to 1000 times greater than the width of the biolistic barrel. The length of the elongated structure may vary, and in some instances ranges from 1 to 1000 cm, such as 2 to 500 cm, including 5 to 50 cm, for example 5 to 20 cm. In certain instances, the length of the elongated structure is 50 cm or less, such as 40 cm or less, including 30 cm or less, for example 20 cm or less. The width of the elongated structure may also vary, and in some instances ranges from 0.5 to 10 cm, such as 0.75 to 5 cm, including 1 to 2 cm. In some instances, the cross-sectional area of the elongated structure ranges from 0.25 to 100 cm², such as from 0.5 to 25 cm², such as 1 to 5 cm². The biolistic barrel may be configured to have a size that facilitates the ease of operation of the biolistic device by a user. For example, the biolistic barrel may be configured to fit comfortably within a user's hand during operation. In addition, the biolistic barrel may be configured to have a weight that facilitates the ease of operation and manipulation of the biolistic device by a user. For instance, the biolistic barrel may have a weight of 250 g or less, such as 100 g or less, including 75 g or less.

The cross-section of the biolistic barrel and lumen extending therethrough may be any desired shape, including, but not limited to, square, rectangular, triangular, circular, elliptical, and the like. In some instances, the cross-sectional shape of the elongate body and included lumen is circular, defining an inner and outer diameter of the biolistic barrel. In such embodiments, the thickness of the walls of the biolistic barrel is defined as the difference between the inner and outer diameter. The inner diameter of the barrel may vary, ranging in some instances from 0.5 to 10 mm, such as from 0.75 to 5 mm, and including, from 1 to 2 mm. The outer diameter of the barrel may vary, ranging in some instances 0.5 to 10 cm, such as 0.75 to 5 cm, including 1 to 2 cm. In some instances, the elongated structure may have a wall thickness ranging from 0.5 to 10 mm, such as from 1 to 5 mm, including from 2 to 4 mm, for example from 2.5 to 3.5 mm. The cross-sectional diameter of the lumen and elongate body may remain constant along the length of the elongate body in some embodiments. In other embodiments, the diameter of the lumen may vary along the length of the elongate body.

Biolistic barrels according to embodiments of the present disclosure have a proximal end and a distal end, where the proximal end and the distal end of the elongated body are arranged on opposing ends of the biolistic barrel. For example, in some embodiments, the proximal end and distal end of the biolistic barrel are arranged at opposing ends of the longitudinal axis of the biolistic barrel. The term “proximal end”, as used herein, refers to the end of the biolistic barrel that is closer to a gas source which accelerates gas and biolistic particles through the biolistic barrel.

Embodiments of the biolistic barrel include an elevated pressure gas inlet at the proximal end. The elevated pressure gas inlet may include, but is not limited to, a hole, aperture, valve, or other type of opening that may allow the passage of gas into the lumen of biolistic barrel. In certain embodiments, the elevated pressure gas inlet is in fluid communication with the lumen of the biolistic barrel. The cross-section of the proximal end elevated pressure gas inlet may be any desired shape, including, but not limited to, square, rectangular, circular, elliptical, and the like. In certain embodiments, the cross-section of the proximal end elevated pressure gas inlet is circular in shape. In various embodiments, the cross-sectional diameter of the proximal end elevated pressure gas inlet may be the same as or vary from the cross-sectional diameter of the biolistic barrel. In certain embodiments, the proximal end elevated pressure gas inlet has an inner diameter that is substantially the same as the diameter of the distal end particle outlet, e.g., where the magnitude of any difference in diameter is 10% or less, such as 5% or less including 2.5% or less.

The biolistic barrels can be configured to attach to any device that accelerates gas and biolistic particles. In certain embodiments, the proximal end elevated pressure gas inlet is configured such that it can attach to an outlet of a device that accelerates gas, where the device may accelerate gas or a combination of gas and biolistic particles, e.g., depending on whether the barrel itself includes an integrated particle cartridge. Any type of connector can be used to attach a device at the proximal end elevated pressure gas inlet of the biolistic barrel. In certain embodiments, the proximal end elevated pressure gas inlet has interlocking elements which operate in conjunction with mating elements on the device to be attached. In certain embodiments, the proximal end elevated pressure gas inlet has interlocking elements which operate in conjunction with mating elements on an open end of a device that accelerates gas and biolistic particles. Any convenient interlocking elements may be employed, e.g., snap fit elements, screw threads, etc. Alternatively, the proximal end elevated pressure gas inlet may be configured such that an open end on a device can pressure fit inside the proximal end elevated pressure gas inlet, such that the open end and proximal end elevated pressure gas inlet are in fluid communication. An example of such a configuration is where the walls of the biolistic barrel and proximal end elevated pressure gas inlet are made of a rigid material, where the inner diameter of the proximal end elevated pressure gas inlet is slightly larger than the outer diameter of an open end, such as a nozzle, on a device. Where the open end of the device is made of an elastomeric material, the portion of the biolistic barrel containing the proximal elevated pressure gas inlet can be pressure fit into the open end of a device by stretching the open end of the device. When any stretching force is removed from the open end of the device, the open end of the device will then comply with the rigid portion of the biolistic barrel containing the proximal end elevated pressure gas inlet such that the device and biolistic barrel are securely attached.

The elevated pressure gas inlet may be configured to apply low positive pressure sufficient to keep the exit port (e.g., of the barrel or a needle/catheter operatively attached thereto) clear of any fluid which may be present in the target tissue so that the biolistic particles have a clear trajectory into the target tissue(s) and/or cell(s). A pressurized gas source can be configured so that during operation a low pressure gas enters the lumen of the biolistic barrel via the elevated pressure gas inlet. For instance, gases such as nitrogen, argon, xenon, carbon dioxide, air, helium, etc. may be used with embodiments of the present disclosure. In certain embodiments, the elevated pressure gas inlet facilitates maintaining a positive gas pressure within the lumen of the biolistic barrel to prevent fluid (i.e., vitreous humor, aqueous humor, blood, lymph fluid, cerebrospinal fluid, peritoneal fluid, pleural fluid, saline, Ringer's solution, water, etc.) from entering the distal end particle outlet during operation. In certain embodiments, the elevated gas pressure inlet provides a constant positive pressure of 1 psi or lower, e.g., 0.5 psi or lower, such as 0.25 psi or lower. In certain embodiments, the elevated pressure gas inlet is in fluid connection with a gas source during operation, allowing gas to enter the biolistic barrel and maintain a desired positive pressure in the barrel, e.g., a pressure that is not too great, such as a pressure ranging in the parameters provided above. In some instances, the elevated pressure gas inlet is operated in conjunction with the pressure-reducing element, e.g., one or more blow-off valves, in order to maintain a desired relatively constant positive pressure.

As described above, the biolistic barrels also include a distal end particle outlet located at the end opposing the proximal end of the elongated body. The term “distal end”, as used herein, refers to the end of the biolistic barrel that is closer to a desired target site. The distal end particle outlet may include an outlet that allows the passage of gas and biolistic particles from the lumen of the biolistic barrel to a desired target site. The cross-section of the distal end particle outlet may be any desired shape, including, but not limited to, square, rectangular, triangular, circular, elliptical, and the like. In certain embodiments, the distal end particle outlet is circular in shape. In various embodiments, the cross-sectional diameter of the distal end particle outlet may be the same as or vary from the cross-sectional diameter of the biolistic barrel. In certain embodiments, the distal end outlet has an inner diameter ranging from 0.1 mm to 10 mm, such as from 0.2 mm to 5 mm.

The biolistic barrels can be configured to attach to an additional delivery device (e.g., an in vivo delivery device), such as a rigid catheter, flexible catheter, tubing, needle, or cannula to facilitate precise delivery of gas and biolistic particles to a target site in vitro or in vivo. In certain embodiments, the distal end particle outlet is configured to attach to a needle such that the lumen of the needle is in fluid communication with the lumen of the biolistic barrel. Any type of connector can be used to attach a needle to the distal end particle outlet of the biolistic barrel. In certain embodiments, the distal end particle outlet has interlocking elements which operate in conjunction with mating elements attached to a needle. As described above, examples of interlocking elements include snap fit elements, pressure fit elements and screw threads. In certain embodiments, the distal end particle outlet may be configured such that it can fit inside a connecting element at one end of a needle so that the needle and distal end particle outlet are in fluid communication. An example of such a configuration is where a needle has a connecting element at one end including a compliant opening. The compliancy is sufficient to impart to the connecting element the ability to pressure fit the distal end particle outlet when a portion of the biolistic barrel containing the distal end particle outlet is inserted through the compliant opening of the connecting element. Once the distal end particle outlet is inserted into the compliant opening, the compliant opening of the connecting element can conform to the shape and diameter of the distal end particle outlet such that the needle and distal end particle outlet are securely attached in fluid communication.

In certain embodiments, the distal end particle outlet is in fluid communication with an attached standard “Luer” type needle. The needle may be of any size large enough to carry biolistic particles. In some instances, needle size ranges from 7 to 50 gauge, such as 7 to 34 gauge, including 7 to 25 gauge. The needle may extend from the end of the distal end particle outlet at a specified length. In certain embodiments, the needle may extend at a length to facilitate the in vivo delivery of a biolistic particle to a target tissue. In certain embodiments, the needle extends from the distal end particle outlet at a distance of 1 to 50 cm, such as 2 to 25 cm, for example 5 to 10 cm. The needle may be sharp or blunt, and have any convenient tip configuration, e.g., beveled, etc.

The biolistic barrels also include one or more pressure-reducing elements. The pressure-reducing elements may be associated with the biolistic barrel according to any convenient arrangement. In some instances, the pressure-reducing elements are positioned between the proximal end elevated pressure gas inlet and the distal end particle outlet. The pressure-reducing element(s) allow for low pressure gas to exit the barrel at the distal end particle outlet, thus decreasing the probability of damage to a target site by high pressure gas bombardment. As used herein, the phrase “pressure-reducing element” refers to a structure that allows pressure inside the biolistic barrel to be dissipated or decreased. Pressure-reducing elements reduce the pressure inside the biolistic barrel by providing a passageway through which gas pressure can exit the lumen of the biolistic barrel. Of interest are pressure-reducing elements whose fluid passageway can be modified, i.e., altered, so as to increase or decrease the flow capacity of the passageway.

In certain embodiments, the pressure-reducing elements are valves. The term “valve” refers to a device for controlling the flow of a fluid through a valve passageway. A valve may be configured as an adjustable valve, such that the valve controls the flow of the fluid by fully or partially opening, or fully or partially closing the valve passageway to the flow of the fluid through the passageway. In certain embodiments, the valve is configured to regulate the flow of the fluid through the valve passageway by increasing or decreasing the flow capacity of the passageway as the fluid is flowing through the passageway. For example, the valve may be configured to adjustably increase or decrease the cross-sectional area of the valve passageway through which the fluid flows. As such, a valve may be configured to provide a level of control over the flow capacity of the valve passageway that is greater than the mere presence of openings or apertures in the passageway. In certain embodiments, the valve includes a valve member. The valve member may be configured as an adjustable valve member, such that the valve member is configured to regulate the flow of the fluid through the valve passageway by increasing or decreasing the flow capacity of the passageway as the fluid is flowing through the passageway, for example, by increasing or decreasing the cross-sectional area of the valve passageway through which the fluid flows. In some cases, the valve member is configured in a closed position, such that substantially no fluid can flow through the valve passageway. In other instances, the valve member may be configured in an open position, such that fluid can flow through the valve passageway. The valve member may be configured in a fully open position, where the flow capacity of the valve passageway is at a substantial maximum, or the valve member may be configured in a partially open position (e.g., a partially closed position), where the flow capacity of the passageway is between the substantial maximum and the flow capacity when the valve member is in a closed position (e.g., substantially zero). Any convenient type of valve may be employed in these embodiments, including but not limited to solenoid valves, blow-off valves, etc. Depending on the nature of the valve, the configuration of the valve relative to the barrel may vary. For example, the valve may be positioned to one side of the barrel and therefore in communication with one side of the lumen of the barrel. In other embodiment, the valve may be integrated with and surround the lumen of the barrel (e.g., where the valve is a solenoid valve).

In some instances, the pressure-reducing elements are blow-off valves. The term “blow-off valve”, as used herein, refers to a device that can dissipate fluid pressure within a body by controlling the flow of a fluid exiting the body. In certain embodiments, the blow-off valves of the present invention are elongate structures including a first end and a second end, where the first end and second end are arranged on opposing ends of the blow-off valve. The first end of the blow-off valve includes an opening that is in fluid communication with the lumen of the biolistic barrel. The second end of the blow-off valve includes an opening that is in fluid communication with the atmosphere. Of interest are blow-off valves configured such that, during operation, gas passing through the lumen of the biolistic barrel enters the blow-off valve through the first end and then exits the second end into the atmosphere. In certain embodiments, the blow-off valves are in fluid communication with the lumen of the biolistic barrel and allow gas to exit the lumen of the barrel, thus dissipating the gas pressure inside the lumen of the biolistic barrel. Due to the presence of blow-off valve(s) in these embodiments, the pressure of the gas stream exiting the distal end particle outlet of the biolistic barrel is lower than the gas pressure carrying biolistic particles into the proximal end elevated pressure gas inlet. Embodiments of the biolistic barrel may include at least one blow-off valve having the ability to decrease gas pressure within the biolistic barrel by a value of 0.25 psi or more, such as 2.5 psi or more, such as 5 psi or more, such as 10 psi or more, such as 25 psi or more, such as 50 psi or more, including 100 psi or more. In certain embodiments, during operation, the ratio of gas pressure exiting at the distal end particle outlet to the gas pressure entering at the proximal end elevated pressure gas inlet ranges from 1 to 1000, such as from 1 to 100, for example, from 1 to 20, including from 1 to 10. In some instances, the one or more pressure reducing elements (e.g., one or more blow-off valves) are configured so that a gas stream has a positive pressure exiting the distal end outlet of the biolistic barrel of 10 psi or less, such as 5 psi or less, including 2.5 psi or less, for example 1 psi or less, or 0.5 psi or less, or 0.25 psi or less. For example, the one or more blow-off valves may have a threshold release pressure, such that the blow-off valve is configured to release gas from the lumen of the biolistic barrel to the surrounding atmosphere when the gas pressure inside the lumen of the biolistic barrel is 10 psi or more, such as 5 psi or more, including 2.5 psi or more, for example 1 psi or more, or 0.5 psi or more, or 0.25 psi or more. As such, the blow-off valves may be configured to have a threshold release pressure of 10 psi or less, such as 5 psi or less, including 2.5 psi or less, for example 1 psi or less, or 0.5 psi or less, or 0.25 psi or less.

In certain embodiments, the flow of gas exiting the biolistic barrel via the blow-off valve can be manually altered. For example, the opening at the second end of the blow-off valve can be obstructed to decrease the flow of gas exiting the second end into the atmosphere. Any convenient mechanism of obstructing the second end of a blow-off valve may be employed. Blow-off valves of interest include, but are not limited to, variable diaphragm, reed valve, and plug based blow valves. For example, in certain embodiments, a plug is inserted into an opening at the second end of a blow-off valve. The term “plug”, as used herein, refers to a structure that may be used to obstruct an opening or aperture. In certain embodiments, a plug can be inserted into the second end of the blow-off valve to obstruct the opening at the second end of the blow-off valve. The plug can have interlocking elements which operate in conjunction with mating elements at the second end of the blow-off valve. As described above, examples of interlocking elements include snap fit elements, pressure fit elements and screw threads. In certain embodiments a plug may be configured such that it can fit the second end of a blow off valve through a pressure fit connection. An example of such a configuration is where the plug is a compliant structure. The compliancy is sufficient to impart to the plug the ability to pressure fit the opening at the second end of a blow-off valve when the plug is inserted through the opening. Once the plug is inserted into the opening, the plug can conform to the shape and diameter of the second end of the blow-off valve such the opening at the second end is closed. In certain embodiments, the cross-sectional diameter of a plug has a substantially similar diameter as the second end of the blow-off valve, such that when the plug is inserted, the second end of the blow-off valve is substantially closed. By “substantially” is meant 80% or more, 85% or more, 90% or more, such as 95% or more, including 98% or more, for example 99% or more. In yet other embodiments, the plug does not have substantially the same diameter as the valve, e.g., where the plug has a diameter that is 75% or less, such as 50% or less, including 25% or less, e.g., 5% or less, of the diameter of the second end. In certain embodiments, a plug is made of a plastic or polymeric material or metal, e.g., stainless steel.

Biolistic barrels can include a plurality of blow-off valves positioned at any convenient location, for example between the proximal end elevated pressure gas inlet and distal end particle outlet. Certain embodiments of the biolistic barrels include 1, 2, 3, 4, 5, 6, 7, 8, 9, but not more than 10 blow-off valves positioned between the proximal end elevated pressure gas inlet and distal end particle outlet. In some cases, having two or more blow-off valves in fluid communication with the lumen of a biolistic barrel facilitates a decrease in gas pressure exiting the distal end outlet of the biolistic barrel. Arrangement of the blow-off valves may be varied as desired. The blow-off valves may protrude from the biolistic barrel at any angle, as desired. In certain embodiments, a blow-off valve can protrude at an angle ranging from 1 to 90 degrees relative to the longitudinal axis of the barrel, such as 5 to 75 degrees relative to the longitudinal axis of the barrel, for example 10 to 45 degrees relative to the longitudinal axis of the barrel. In certain embodiments, the blow-off valves may extend perpendicular to the longitudinal axis of the biolistic barrel. In certain embodiments, the blow-off valves may surround the main gas flow path. In some instances, the blow-off valves are positioned at 2 cm or closer to the distal end particle outlet, such as at 1 cm or closer to the distal end particle outlet. In some instances, biolistic barrels include an array of blow-off valves positioned between the proximal end elevated pressure gas inlet and distal end particle outlet. For instance, embodiments of the biolistic barrels can include an array of blow-off valves arranged with a desired spacing.

In certain embodiments, the biolistic barrel includes one pressure-reducing element, such as one blow-off valve. The single blow-off valve can be positioned at any convenient location along the biolistic barrel, for example between the proximal end elevated pressure gas inlet and distal end particle outlet. In some cases, the single blow-off valve is a high volume pressure-reducing element. By “high-volume” is meant that the single blow-off valve is configured to decrease gas pressure within the biolistic barrel by an amount substantially the same as or greater than the plurality of blow-off valves (e.g., two or more blow-off valves) described above. In certain instances, embodiments that include a single high-volume blow-off valve are less complex (e.g., include fewer parts) than embodiments that include a plurality of blow-off valves, which, in some cases, may facilitate a simplification in manufacturing, a reduction in the overall cost of the device, and/or a reduction in the overall dimensions (e.g., the width) of the biolistic barrel. Similar to the plurality of blow-off valves described above, the single blow-off valve may protrude from the biolistic barrel at any angle, as desired. In certain embodiments, the single blow-off valve can protrude at an angle ranging from 1 to 90 degrees relative to the longitudinal axis of the barrel, such as 5 to 75 degrees relative to the longitudinal axis of the barrel, for example 10 to 45 degrees relative to the longitudinal axis of the barrel. In certain embodiments, the single blow-off valve may extend perpendicular to the longitudinal axis of the biolistic barrel.

In some embodiments, the biolistic barrels further include a low pressure gas inlet. This low pressure gas inlet may be associated with the barrel via any convenient configuration. In some instances, this low pressure gas inlet is positioned between the proximal end elevated pressure gas inlet and the distal end particle outlet. In various embodiments, the low pressure gas inlet can be a hole, aperture, nozzle or valve in fluid communication with the lumen of the biolistic barrel. In certain embodiments, the low pressure gas inlet is a nozzle in fluid communication with the lumen of the biolistic barrel. A first end of the nozzle includes an opening, such as a hole, such that gas from a pressurized gas source can enter the first end of the nozzle and then enter the biolistic barrel through a second end of the nozzle that is continuous with the lumen of the biolistic barrel. Through the use of any convenient connector and pressurized gas source coupler, a pressurized gas source can be configured so that during operation a low pressure gas enters the lumen of the biolistic barrel via the low pressure gas inlet. For example, the low pressure gas inlet may include at least one valve (e.g., a low pressure gas inlet valve). The valve may be configured to adjust the pressure of the gas source that enters the lumen of the biolistic barrel through the low pressure gas inlet. In some instances, the valve may be adjustable to allow gas to enter the biolistic barrel and maintain a desired positive pressure in the biolistic barrel.

In certain embodiments, the low pressure gas inlet facilitates maintaining a positive gas pressure within the lumen of the biolistic barrel to prevent fluid (i.e., vitreous humor, aqueous humor, blood, lymph fluid, cerebrospinal fluid, peritoneal fluid, pleural fluid, saline, Ringer's solution, water, etc.) from entering the distal end particle outlet during operation. In certain embodiments, the low pressure gas inlet is in fluid connection with a gas source during operation, allowing gas to enter the biolistic barrel and maintain a desired positive pressure in the barrel, e.g., a pressure that is not too great, such as a pressure ranging in the parameters provided above. In some instances, the low pressure gas inlet is operated in conjunction with the pressure-reducing element, e.g., one or more blow-off valves, in order to maintain a desired relatively constant positive pressure.

The pressure-reducing elements and low pressure gas inlet may be integrated into the biolistic barrel in a variety of different configurations. Integrated configurations include configurations where the at least one pressure-reducing element and low pressure gas inlet are fixed relative to the distal end particle outlet. Alternatively, at least one pressure-reducing element and low pressure gas inlet may include separate structures capable of being attached to the elongate structure of the biolistic barrel after it has been fabricated. In certain embodiments, holes can be drilled into the elongate structure of the biolistic barrel and the at least one pressure-reducing element and low pressure gas inlet can be inserted into the holes to be in fluid communication with the lumen of the biolistic barrel. Specific configurations of interest are further described below in connection with the figures.

In some embodiments, the elongate body of the biolistic barrel includes at least one pressure-reducing element. In such embodiments, the one or more pressure-reducing elements and low pressure gas inlet can be integrated into the body of the biolistic barrel during the fabrication process of the biolistic barrel. Within the elongate body of the biolistic barrel, at least one pressure-reducing element and low pressure gas inlet may be constructed from any material that is durable enough to withstand internal gas pressure ranges (e.g. described above) produced by the biolistic device to which the barrel is operatively coupled. In certain embodiments, the elements of the present invention can be constructed of a metal (e.g., steel, stainless steel, titanium, aluminum, etc., and alloys thereof), ceramic material, plastics (e.g., Delrin, thermoplastics, thermoset plastics, etc.), or polymeric materials (e.g., polytetrafluoroethylene, polyimide, etc.). In a certain embodiment, the biolistic barrel is made of a light metal, such as aluminum alloy. Alternatively, the biolistic barrel can be made of a biocompatible metal, for example, stainless steel. The material should allow the biolistic barrel to withstand the bombardment of biolistic particles into the barrel's inner wall. Any convenient fabrication protocol may be employed in the fabrication of the biolistic barrel and its elements, such as molding, machining, etc.

In some instances, the biolistic barrel further includes a cartridge holder. The cartridge holder may be positioned between the proximal end elevated pressure gas inlet and the distal end particle outlet of the biolistic barrel. The cartridge holder is defined by a chamber with one or more fluid passageways in fluid communication with the lumen of the biolistic barrel (e.g., in fluid communication with the proximal end elevated pressure gas inlet and the distal end particle outlet of the biolistic barrel). Additional aspects of the cartridge holder are described in more detail below.

The biolistic barrels may be disposable or reusable. As such, the biolistic barrels may be entirely reusable (e.g., be multi-use devices) or be entirely disposable (e.g., where all components of the device are single-use). In some instances, the biolistic barrels can be entirely reposable (e.g., where all components can be reused a limited number of times). Each of the components of the device may individually be single-use, of limited reusability, or indefinitely reusable, resulting in an overall device or system including components having differing usability parameters.

An embodiment of the biolistic barrel is shown in FIG. 1. The biolistic barrel 100 includes a proximal end elevated pressure gas inlet 102, a distal end particle outlet 104, three pressure-reducing elements identical to blow-off valve 106, and a low pressure gas inlet 108 positioned between the proximal end elevated pressure gas inlet and the distal end particle outlet. In the exhibited configuration, the three blow-off valves extend at a 90 degree angle from the lumen of the biolistic barrel. The flow stream of gas entering the proximal end elevated pressure gas inlet 102 is shown as an arrow 101. In the embodiment shown in FIG. 1, the biolistic barrel 100 includes a lumen of substantially constant diameter that runs along the longitudinal length of the barrel. By “substantially” is meant 80% or more, 85% or more, 90% or more, such as 95% or more, including 98% or more, for example 99% or more.

The biolistic barrels can be configured to be attached to a source of gas. The biolistic barrel can include a fitting 103 configured to attach the barrel to a gas source or gas source coupler. The fitting can be shaped as cylinder with a diameter smaller than that of the biolistic barrel lumen. The proximal end elevated pressure gas inlet 102 can be configured for attachment to a biolistic device through any convenient connection technique, such as a snug-fit connection, a press-fit connection or a threaded-fit connection. When configured for insertion into a biolistic device, the fitting 103 can correspond to the inner diameter of a nozzle on gas source or gas source coupler. In the exhibited embodiment, the biolistic barrel can be attached to the gas source or gas source coupler by inserting the fitting 103 in an aperture of substantially similar diameter within the gas source/coupler.

In some embodiments, biolistic barrels include one or more integrated functionalities beyond that shown in the embodiment of FIG. 1. For instance, biolistic barrels may include one or more of: an integrated biolistic particle cartridge, a handle, a flexible tubing, an adapter to interface with a biolistic device, etc. For example, FIG. 5 provides an illustration of an embodiment of a biolistic barrel 500 that includes, in addition to the pressure-reducing element (as present in three blow-off valves 550) and low pressure gas inlet 540, an integrated biolistic particle cartridge/handle 530, a flexible tubing 520, and an adapter 510 for operatively coupling the biolistic barrel to a biolistic delivery device. As shown in FIG. 5, at the proximal end of biolistic barrel 500 includes an adapter 510 configured to interface with a biolistic device, such as a gene gun. The adapter 510 may be a universal adapter, or a specific adapter for a specific type of biolistic device, as desired. Operatively coupled in fluid communication to the adapter 510 is a flexible tubing 520. The length of this tubing 520 may vary; in some instances the length ranges from 1 cm to 1000 cm, such as 5 cm to 50 cm, e.g., 10 cm to 25 cm. Operatively coupled in fluid communication to the flexible tubing 520 is a biolistic particle cartridge/handle 530. The biolistic particle cartridge/handle 530 is analogous to the cartridge as described above, with the cartridge being present in a housing that serves as a handle configured for manipulation (e.g., handheld, stereotactically, micromanipulator mounted) by a user. The cartridge/handle 530 is, in turn, operatively coupled in fluid communication to a barrel portion that includes three blow-off valves 550 and a low pressure gas inlet 540, where these elements are as described above. In some embodiments, the distal end of the barrel is a Luer connector 560 which is coupled to a delivery needle or cannula 570.

Embodiments of the biolistic barrel are also shown in FIG. 6, specifically FIGS. 6A and 6B. The biolistic barrel 600 includes a pressurized gas source coupler 604, a handle 606, a distal end particle outlet 608, a needle 610, a pressure-reducing element (e.g., a blow-off valve) 612, and a cartridge holder 614. The cartridge holder 614 is a rotatable cartridge holder that is configured to successively rotatably position the cartridges in fluid communication with the lumen of the biolistic barrel. The biolistic barrel is configured to accept a low pressure gas stream through the pressurized gas source coupler 604 an into the biolistic barrel to maintain a constant positive pressure, which prevents fluid (i.e., vitreous humor, aqueous humor, blood, lymph fluid, cerebrospinal fluid, peritoneal fluid, pleural fluid, saline, Ringer's solution, water, etc.) from entering through the distal end particle outlet 608. The low pressure gas stream may be provided through a low pressure gas inlet (not shown), which in some embodiments is positioned upstream from the pressurized gas source coupler 604. FIG. 6A also depicts actuator 602, which is described in further detail below in relation to embodiments of the biolistic devices.

### Biolistic Devices

As summarized above, embodiments of the present disclosure include biolistic devices. Biolistic devices are systems that employ a stream of gas to accelerate biolistic particles through a lumen in a biolistic barrel. Of interest are biolistic devices that can be configured to deliver a molecule to a target site under low pressure. In some instances, the biolistic device includes a pressurized gas source coupler, a biolistic barrel, a cartridge holder positioned between the pressurized gas source coupler and the biolistic barrel, and an actuator for actuating the biolistic device. In yet other instances, the biolistic device may lack the cartridge holder, e.g., where the particles/cartridge are integrated into the barrel, e.g., as shown in the embodiment of FIG. 5, described in greater detail below.

As summarized above, the biolistic devices include a pressurized gas source coupler. A pressurized gas source coupler can be any apparatus, machine, conduit, or device that produces a desired gas flow when coupled with a pressurized gas source. In certain embodiments, the pressurized gas source coupler can include a conduit that carries gas from a pressurized gas source to the barrel, and in some instances to a cartridge holder positioned between the pressurized gas source coupler and the biolistic barrel. The pressurized gas source coupler and optional cartridge holder are in fluid communication with the lumen of the biolistic barrel. The conduit of the pressurized gas source coupler can be made of any convenient material that is able to carry a gas from a pressurized gas source. Examples of convenient materials include, but are not limited to, polyethylene, copper, steel, aluminum, and polyvinyl chloride.

A pressurized gas source may include any number of devices to provide gas in the biolistic devices. Any convenient gas source may be used in embodiments of the biolistic devices. Gas sources may produce regulated gas flow, but this is not required in aspects of the biolistic devices. The pressurized gas source coupler, in conjunction with a gas source, may provide a number of gases to the conduit in various embodiments. For instance, gases such as nitrogen, argon, xenon, carbon dioxide, air, helium, etc. may be used with embodiments of the present disclosure. The gas need not be inert and should be capable of carrying a sufficient quantum of energy or heat. Any gases that contain these characteristic properties may also be used with the biolistic devices.

The biolistic devices also include biolistic barrels. As described above, the biolistic barrels include one or more pressure-reducing elements positioned between the proximal end gas inlet and the distal end particle outlet. The pressure-reducing elements allow for low pressure to exit the barrel at the distal end particle outlet, thus decreasing the likelihood/probability of damage to a target site by high pressure gas bombardment. Pressure-reducing elements reduce the pressure inside the biolistic barrel by opening or partially exposing the lumen inside the biolistic barrel. As described above, the pressure-reducing elements may include blow-off valves. Certain embodiments of the biolistic barrels include 2, 3, 4, 5, 6, 7, 8, 9, but not more than 10 blow-off valves positioned between the proximal end elevated pressure gas inlet and distal end particle outlet. In some instances, the biolistic device is configured so that a gas stream entering the biolistic barrel through the proximal end has a positive pressure ranging from 10 to 50 psi, and a positive pressure exiting the distal end outlet of the biolistic barrel of 10 psi or less, such as 5 psi or less, including 2.5 psi or less, for example 1 psi or less, or 0.5 psi or less, or 0.25 psi or less.

As described above, the biolistic barrels of the biolistic devices further include a low pressure gas inlet that in some instances may be positioned between the proximal end elevated pressure gas inlet and the distal end particle outlet. The low pressure gas inlet of the biolistic barrel facilitates maintaining a positive gas pressure within the lumen of the biolistic barrel to prevent fluid from entering the distal end particle outlet. In certain embodiments, the low pressure gas inlet is in fluid connection with a gas source allowing gas to enter the biolistic barrel at a desired pressure, e.g., 1 psi or lower, such as 0.5 psi or lower, including 0.25 psi or lower.

In certain embodiments, the low pressure gas inlet is positioned upstream from the proximal end elevated pressure gas inlet. By “upstream” is meant that the element of interest is closer to the gas source which accelerates gas and biolistic particles through the biolistic barrel. The low pressure gas inlet may be positioned downstream from an actuator (actuators are described in more detail below). By “downstream” is meant that the element of interest is further away from the gas source. For example, the low pressure gas inlet may be positioned upstream from the proximal end elevated pressure gas inlet and downstream from the actuator, such that the low pressure gas inlet is positioned between the proximal end elevated pressure gas inlet and the actuator. In these embodiments, the low pressure gas inlet may be associated with the barrel via any convenient configuration, such that the low pressure gas inlet is in fluid communication with the proximal end elevated pressure gas inlet and the lumen of the biolistic barrel. In some instances, the low pressure gas inlet is configured to provide a pressurized gas from a pressurized gas source to the proximal end elevated pressure gas inlet and then to the lumen of the biolistic barrel, which is in fluid communication with the proximal end elevated pressure gas inlet. The pressurized gas source can be configured so that during operation a low pressure gas enters passes through the low pressure gas inlet and then through the proximal end elevated pressure gas inlet and into the lumen of the biolistic barrel. As described above, in certain instances, the low pressure gas inlet facilitates maintaining a positive gas pressure within the lumen of the biolistic barrel to prevent fluid (i.e., vitreous humor, aqueous humor, blood, lymph fluid, cerebrospinal fluid, peritoneal fluid, pleural fluid, saline, Ringer's solution, water, etc.) from entering the distal end particle outlet during operation.

In some instances, the biolistic devices further include a cartridge holder. The cartridge holder may be positioned between the pressurized gas source coupler and the biolistic barrel. In some instances, as described above, the cartridge holder is included in the biolistic barrel. For example, the cartridge holder may be positioned between the proximal end elevated pressure gas inlet and the distal end particle outlet of the biolistic barrel. The cartridge holder is defined by a chamber with one or more fluid passageways in fluid communication with the pressurized gas source coupler and the proximal end elevated pressure gas inlet of the biolistic barrel. As with the biolistic barrels described above, the cartridge holder can be constructed from any material that is durable enough to withstand the internal gas pressures generated by the biolistic device, e.g., as described above. In certain embodiments, the cartridge holder can be constructed of metal (e.g., steel, stainless steel, titanium, aluminum, etc., and alloys thereof), ceramic material, plastic (e.g., Delrin, thermoplastics, thermoset plastics, etc.), and/or polymeric materials (e.g., polytetrafluoroethylene, polyimide, etc.).

The cartridge holder further contains one or more housings for cartridges in fluid communication with the fluid passageways of the cartridge holder. The housings in the cartridge holder can be designed to hold cartridges that carry biolistic particles. Cartridges that can be employed in the biolistic devices include, for example, hollow elongate tubes in fluid communication with the fluid passageway of the cartridge holder. In certain embodiments, the hollow elongate tubes of the cartridges are made of plastic tubing which allows biolistic particles to adhere to the inner surface of the hollow elongate tube of the cartridge. The pressure of a gas stream is used to dislodge the biolistic particles from the cartridge within the cartridge holder. Further non-limiting examples of cartridges which can be employed in the present invention are described in U.S. Pat. Nos. 6,004,287; 6,436,709; and 7,638,332.

As described above, in some cases, the cartridge holder includes one or more housings configured to hold cartridges that carry biolistic particles. The cartridge holder may be configured to position one of the cartridges in fluid communication with the lumen of the biolistic barrel while positioning the other cartridges in the cartridge holder away from the lumen of the biolistic barrel. For example, the cartridge holder may be configured to rotatably position the cartridges in the cartridge holder in sequential order in fluid communication with the lumen of the biolistic barrel. In these embodiments, the cartridge holder may position the first cartridge in fluid communication with the lumen, then may be rotated such that the second cartridge is positioned in fluid communication with the lumen. Subsequent rotations of the cartridge holder position the third and successive cartridges in fluid communication with the lumen. In some embodiments, the cartridge holder is configured to hold 3 or more, such as 4 or more, including 5 or more, or 6 or more, or 7 or more, or 8 or more, or 9 or more, or 10 or more cartridges. For instance, the cartridge holder may be configured to hold 6 cartridges, such that the cartridges may be successively rotatably positioned in fluid communication with the lumen of the biolistic barrel as described above. In certain embodiments, the cartridge holder includes one or more alignment guides on the exterior of the cartridge holder, such as, but not limited to, a detent, a notch, a tab, a groove, a guide post, etc., which may facilitate alignment of the cartridges with the lumen of the biolistic barrel.

In certain embodiments, biolistic particles include, but are not limited to, elemental particles of a heavy metal such as gold, silver, or tungsten. The size of the biolistic particles may vary, and are in some instances on the micrometer or nanometer scale, e.g., ranging from 10 nm to 10 μm. The biolistic particles can be coated with a material before they are delivered to the target site. In certain embodiments, the elemental particle can be coated with an active agent. In certain embodiments, the elemental particle can be coated with a polymer such as deoxyribonucleic acid (DNA), ribonucleic acid (RNA), a carbohydrate, a protein, and other biological materials, or mixtures thereof, including synthetic polymeric constructs, e.g., recombinant nucleic acids, non-naturally occurring proteins, peptide nucleic acids, etc. Other active agents that can be delivered by way of the biolistic barrels include, but are not limited to, marker dyes, drugs, complex macromolecules, e.g., dendrimers, and vaccines.

The biolistic devices include an actuator for actuating the biolistic device. Actuators can be any convenient device configured to initiate the delivery of gas to the biolistic device. Examples of convenient actuators include an electromechanical actuator, a mechanical actuator, and a linear actuator. The actuator can be operably connected to a pressurized gas source coupler. By “operably connected” is meant that one structure is in communication (for example, mechanical, electrical, optical connection, or the like) with another structure. In certain embodiments, when the electromechanical actuator is actuated, a pressurized gas source coupler in fluid communication with a pressurized gas source provides a pulse of gas through the proximal end elevated pressure gas inlet and into a cartridge holder in fluid communication with the biolistic barrel. In certain embodiments, the actuator is an electromechanical trigger device. The electromechanical trigger device may be configured to be operated by the hand of a user, such as by squeezing the trigger device between the finger(s) and palm of the user's hand. In other embodiments, the electromechanical trigger device may be configured to be operated by the foot of a user, such as by stepping on a pedal, button or switch. In some instances, a foot-activated trigger device may facilitate operation of the biolistic device by the user by freeing the hands of the user for other tasks, such as positioning and operating the biolistic barrel.

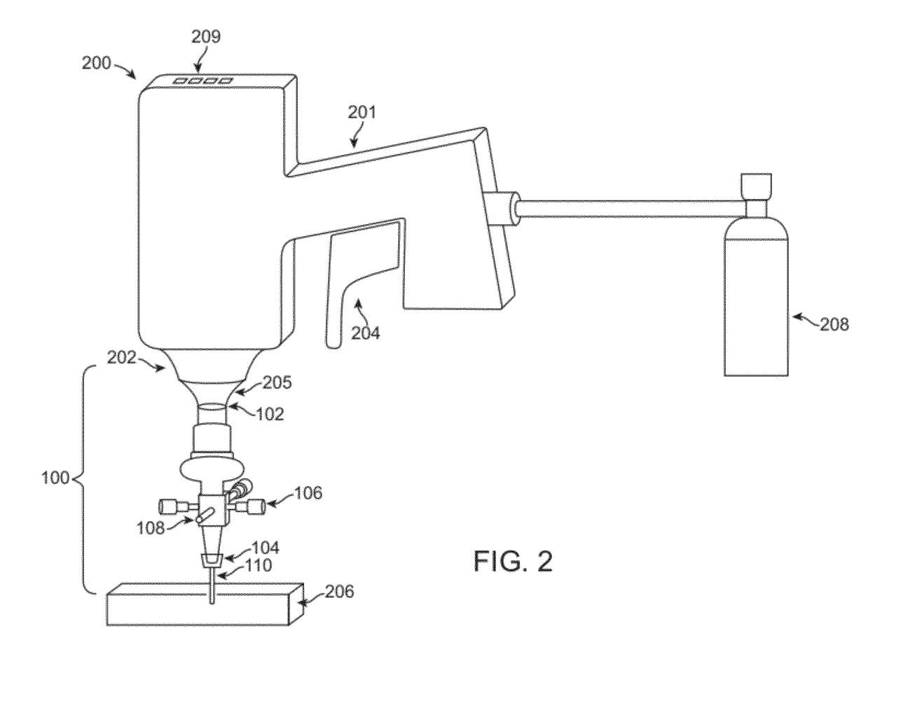

An embodiment of the biolistic device is shown in the apparatus exhibited in FIG. 2. The biolistic device includes a pressurized gas source coupler 202, a biolistic barrel 100 comprising a proximal end elevated pressure gas inlet 102, a distal end particle outlet 104, three pressure-reducing elements identical to blow-off valve 106, and a low pressure gas inlet 108 positioned between the proximal end elevated pressure gas inlet 102 and the distal end particle outlet 104. Gas is provided to a pressure source coupler 202 from a gas source 208 via a gas accelerator 200. During use, the lumen of the biolistic barrel 100 is coupled to a gas source at the proximal end elevated gas pressure inlet 102 of the device. In certain embodiments, the gas source is configured to introduce gas into the proximal end elevated gas pressure inlet 102 under positive pressure, e.g., at a pressure ranging from 10 psi to 50 psi, such that gas is conveyed along the lumen and out the distal end particle outlet. Any convenient gas may be employed in the gas source, such as but not limited to nitrogen, helium, compressed air, etc.

Embodiments of the biolistic barrel 100 can be adapted and attached to any system that accelerates biolistic particles for delivery to a target site via a gas stream. As described above, a connecting means allows the biolistic barrel to be retrofitted into existing biolistic devices. In a certain embodiments, the biolistic barrel may be attached to a “gene gun”. In general, a gene gun is a device that delivers DNA to cells by microprojectile bombardment through high speed delivery. The diameter of the proximal end elevated gas pressure inlet may be sized to match a nozzle or opening on a particular gene gun to which the biolistic barrel will be attached. In a certain embodiments, the diameter of the nozzle of the gene gun and the diameter defined by the sidewalls of the proximal end elevated pressure gas inlet are nearly identical (i.e., within about 10% of the diameter of the nozzle of the gene gun).

An example of a commercially available system that may be used in conjunction with the biolistic device of the present disclosure is the Helios® Gene Gun (Bio-Rad Laboratories, Hercules, Calif.). The embodiment exhibited in FIG. 2 utilizes a Helios® Gene Gun. In the exhibited embodiment, the biolistic device may include an actuator, such as a trigger device, operably connected to the biolistic barrel. By “operably connected” is meant that one structure is in communication (for example, mechanical, electrical, optical connection, or the like) with another structure. In the exhibited embodiment, an actuator, which is in connection with a trigger device 204 on a handle 201, when actuated, sends a signal to allow gas to enter the cartridge holder 205 via the pressure source coupler 202. In certain embodiments, the cartridge is a hollow cylindrical body containing biolistic particles.

In certain embodiments, the biolistic device includes an optional visual display 209. For example, certain embodiments of the present disclosure include a visual display as described above, and in other instances, the biolistic device does not include a visual display. In some instances, the visual display is configured to indicate that gas is entering the cartridge holder 205 from the pressurized gas source 208. In some cases, the visual display is configured to indicate the firing state of the gene gun (e.g., whether the gene gun is ready to fire). Gas flows into and through a cartridge holder 205 dislodging the biolistic particles from the inner surface of the cartridge holder. The biolistic particles are carried by the gas flow to the biolistic barrel 100. The gas/particle mixture enters of proximal end elevated pressure gas inlet 102 and is discharged into the target tissue 206 through the distal end particle outlet 104. In the exhibited embodiment, the biolistic barrel 100 allows for the attachment of a Luer-type needle 110 in fluid communication with the distal end particle outlet 104.

As shown in FIG. 2, the biolistic barrel 100 includes a low pressure gas inlet 108 positioned between the proximal end elevated pressure gas inlet 102 and the distal end particle outlet 104. The low pressure gas inlet 108 allows for constant positive pressure to prevent fluid from entering through the distal end particle outlet of the biolistic barrel. In the exhibited embodiment, the low pressure gas inlet 108 provides a constant positive pressure of 1 psi or lower, e.g., 0.5 psi or lower, such as 0.25 psi or lower. The three pressure-reducing elements identical to blow-off valve 106 positioned between the proximal end elevated pressure gas inlet 102 and the distal end particle outlet 104 moderate the gas pressure within the biolistic barrel during firing of the gene gun and allow for minimal pressure exiting the barrel at the distal end particle outlet, while propelling the particles down the barrel and into the tissue, thus mitigating the potential for damage to the target tissue 206 caused by high-pressure gas bombardment.

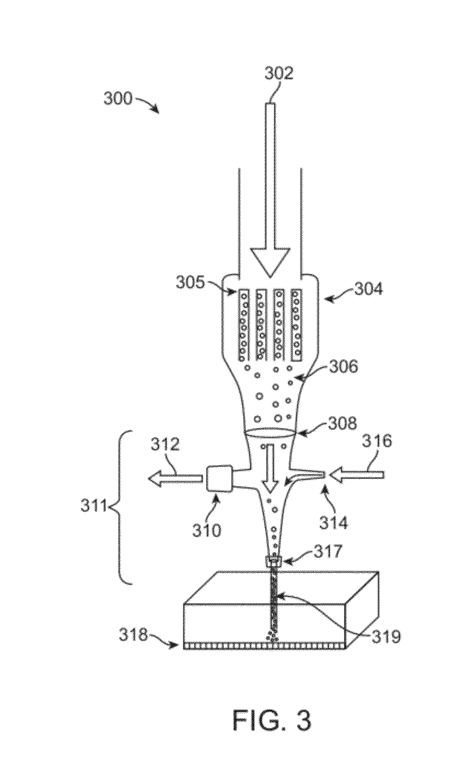

FIG. 3 depicts a cross-section of the biolistic device 300 according to one embodiment. A gas stream 302 enters a cartridge holder 304, releasing a biolistic particle 306 from a cartridge 305 within the cartridge holder. In certain embodiments, the gas stream is helium with a pressure head ranging from 10 to 50 psi. Acceleration and travel of gas occurs along the biolistic barrel of substantially constant diameter, having a smooth inner wall. The biolistic particle 306 is carried by the gas stream into the proximal end elevated pressure gas inlet 308 of the biolistic barrel 311. The biolistic barrel 311 includes a low pressure gas inlet 314 positioned between the proximal end elevated pressure gas inlet 308 and the distal end particle outlet 317. The low pressure gas inlet 314 is configured to allow a gas stream 316 to enter the biolistic barrel 311 and maintain a constant but regulatable positive pressure to prevent fluid (i.e., vitreous humor, aqueous humor, blood, lymph fluid, cerebrospinal fluid, peritoneal fluid, pleural fluid, saline, Ringer's solution, water, etc.) from entering through the distal end particle outlet 317. A pressure-reducing element 310 positioned between the proximal end elevated pressure gas inlet 308 and the distal end particle outlet 317 allows a gas stream 312 to exit the biolistic barrel 311. As shown, the particles leave the biolistic barrel 311 and enter needle 319 which conveys the particles to the target tissue 318. The outgoing gas stream 312 moderates the gas pressure within the biolistic barrel 311 and allows for minimal pressure exiting the biolistic barrel at the distal end particle outlet 317, thus mitigating the potential for damage to the target tissue 318. Where appropriate, particle size and gas velocity can also be altered in various embodiments.

An embodiment of the biolistic device is shown in FIG. 6, specifically FIGS. 6A and 6B. The biolistic device includes a biolistic barrel 600 and an actuator 602. The biolistic barrel 600 includes a pressurized gas source coupler 604, a handle 606, a distal end particle outlet 608, a needle 610, a pressure-reducing element (e.g., a blow-off valve) 612, and a cartridge holder 614. The cartridge holder 614 is a rotatable cartridge holder that is configured to successively rotatably position the cartridges in fluid communication with the lumen of the biolistic barrel. The biolistic device is configured to provide a low pressure gas stream through the pressurized gas source coupler 604 to the biolistic barrel and maintain a constant positive pressure to prevent fluid (i.e., vitreous humor, aqueous humor, blood, lymph fluid, cerebrospinal fluid, peritoneal fluid, pleural fluid, saline, Ringer's solution, water, etc.) from entering through the distal end particle outlet 608. FIG. 6A also depicts actuator 602, which includes an on/off switch 616 and a potentiometer 618. The potentiometer 618 is configured to allow a user to adjust the duration of the gas pulse, e.g., from 0 to 500 msec. The actuator 602 may be operated by a trigger device, such as a foot pedal trigger device (not pictured). FIG. 6C depicts a view of the internals of the actuator 602. In particular, FIG. 6C shows a gas solenoid 620 actuated by trigger network 622, which includes tandem 556 integrated circuit chips and the potentiometer 618. The trigger network 622 is configured to allow the user to control the gas pulse duration. A schematic of the solenoid trigger circuit is shown in FIG. 6D.

### Methods

Also provided are methods of delivering a molecule to a target site using the biolistic device. The methods of delivering a molecule to a target site include (1) positioning a biolistic delivery system (which may be made up of a biolistic delivery device, biolistic barrel and needle/cannula, e.g., as described above) in delivery relationship with the target site and (2) actuating an actuator to deliver the molecule to the target site. The methods result in the delivery of a molecule to a target site under low pressure with substantially little tissue damage. Aspects of the methods are described in greater detail below.

The first step of the subject methods includes positioning a biolistic delivery system in delivery relationship with the target site. The term “delivery relationship”, as used herein, refers to the placement of a component of the biolistic system, e.g., the distal end of a needle operatively coupled to the biolistic barrel, such that it can deliver biolistic particles adjacent to or into the target site. In certain embodiments, the target site is a tissue. For example, the target site can be in vitro or in vivo tissue. In certain embodiments of the methods, the distal end particle outlet can be positioned in a delivery relationship to an in vitro tissue by positioning the distal end particle outlet adjacent to an in vitro tissue, e.g., at a distance ranging from 0.5 mm to 50 mm. In other embodiments, the target site can be in vivo tissue. In certain embodiments, a needle in fluid communication with the biolistic device is introduced into in vivo mammalian tissue such that the biolistic device, via the needle, is in a delivery relationship to the in vivo mammalian tissue. A needle in fluid communication with the biolistic device can be introduced into mammalian tissues in vivo at a suitable depth, e.g., ranging in some instances from 1 mm to 500 mm, such as from 5 mm to 250 mm, for example from 10 mm to 150 mm. For positioning, where desired the system or component(s) thereof can be stereotactically mounted and micro-manipulated with surgical precision.

The biolistic delivery system can be loaded with biolistic particles prior to or after positioning the biolistic delivery system in delivery relationship with a target site. In certain embodiments, the biolistic delivery system is loaded with biolistic particles, e.g., as described above, prior to positioning the biolistic delivery system in delivery relationship with a target site. As reviewed above, the delivery system may include a biolistic device which includes a cartridge holder containing one or more housings for cartridges that can carry biolistic particles. Cartridges that can be employed in the biolistic devices include hollow elongate tubes in fluid communication with the fluid passageway of the cartridge holder. In certain embodiments, biolistic particles can be introduced and adhered to the inner surface of the hollow elongate tube of the cartridge. The biolistic particle-loaded cartridge can then be inserted into a cartridge holder prior to the actuating of the biolistic delivery device.

The second step of the subject methods includes actuating the biolistic delivery device or system that includes the same following the positioning of the biolistic delivery system in delivery relationship with the target site. The actuator is a component of the biolistic delivery system and can be any mechanical device (e.g., a trigger device) operably coupled to a pressurized gas source coupler. In some instances, the trigger device is an electromechanical device which can be actuated manually by a user. Alternatively, the actuator may be actuated automatically by a computer configured to actuate the biolistic delivery system. Once actuated, a pressurized gas source coupler in fluid communication with a pressurized gas source provides a pulse of gas through a cartridge (or similar biolistic particle containing element) and into the proximal end elevated pressure gas inlet in fluid communication with the biolistic barrel. Within the cartridge holder is at least one cartridge containing biolistic particles to be delivered to a target site. The pressure of the gas stream is used to dislodge the biolistic particles from a cartridge within the cartridge holder. The gas pressure used to dislodge the biolistic particles from the cartridge varies according to the depth of tissue penetration desired. In certain embodiments, the biolistic barrel is configured so that a gas stream entering the barrel through the proximal end has a positive pressure ranging from 10 to 50 psi. Once dislodged from the cartridge, the biolistic particles follow the high velocity stream of gas into the biolistic barrel comprising one or more pressure-reducing elements positioned between the proximal end inlet and the distal end particle outlet. In certain embodiments, gas entering the proximal end of the biolistic barrel has a velocity of greater than 200 m/sec, such as greater than 500 m/sec, for example, greater than 1000 m/sec. In certain embodiments, the gas/particle stream exiting the distal end particle outlet has a velocity greater than 200 m/sec, such as greater than 500 m/sec, for example, greater than 1000 m/sec. As described above, the pressure-reducing elements moderate the pressure head created by the gas stream so it is less intense when the gas/particle stream reaches the distal end particle outlet of the biolistic barrel.

### Utility

The biolistic barrels, biolistic devices, kits and methods of delivering a molecule to a target site with the biolistic device find use in a variety of different applications where it is desirable to introduce a molecule to a target site. Essentially any type of surface or material that can be made accessible to the biolistic barrel may be targeted using the biolistic barrels, biolistic devices, methods and kits. In certain embodiments, the biolistic devices may be used to minimize damage to the target during the delivery of a molecule to the target. The target site can be a delicate tissue that is susceptible to damage during biolistic particle bombardment at high pressure. Mammalian tissue is an example of a target site that can be used in embodiments.

The biolistic barrels, biolistic devices, kits, and methods of delivering a molecule to a target site under low pressure can be applied to a variety of targets including, but not limited to a cell, cell cultures, tissues, organs, animals, animal embryos, bacteria, fungi, algae, cell nuclei and organelles such as chloroplasts and mitochondria. Target tissues of interest include the skin, retina, brain, liver, pancreas, spleen, heart, bladder, kidney, and muscle. Other target tissues may include plants, plant cells, seedlings, cultured plant cells, leaves, epidermal tissues, apical meristems, and floral tissues. The biolistic devices and methods of delivering a molecule to a target site can be applied to target tissues in a variety of conditions, including but not limited to in vitro, in vivo, and ex vivo target tissues.

The provided biolistic barrels, biolistic devices, kits, and methods can facilitate the delivery of virtually any molecule, polymer, or active agent to a target site. In certain embodiments, the biolistic devices may find use in the delivery of labeling dyes to a target tissue. In other embodiments, the biolistic devices may find use in the delivery of drugs to a target tissue. General applications of the biolistic barrels, biolistic devices and methods of delivering a molecule to a target site include gene therapy. Gene therapy aims to introduce specific genes into a host to replace defective ones (replacement therapy) or to suppress expression of certain undesirable genes (anti sense therapy). Other potential applications of the biolistic barrels include the research of gene regulation and promoter analysis, in vivo cellular labeling and imaging, and cellular physiology. The biolistic barrels, biolistic devices, kits, and methods can also be used to further understanding of vaccinations, cancer, infectious disease, and wound healing; to generate immune responses in animals; and to assay gene expression and regulation both in vivo an in vitro.

Embodiments of the methods find use in the delivery of foreign molecules (e.g., proteins, such as antibodies; or nucleic acids, such as DNA or RNA,) with surgical precision using catheters/needles, where such delivery may be accomplished in vivo to a living organism with substantially little, if any, tissue damage. Accordingly, embodiments of the present disclosure find use in applications where subcutaneous delivery of foreign molecules to foreign tissue(s) in vivo (i.e., to a living organism) is desired, such as, for example, the transfection of nucleic acids or proteins into cells. Aspects of these embodiments include substantially little, if any, collateral tissue damage.

Embodiments of the subject methods also find use in the multiplex delivery of two or more distinct foreign molecules to one or more target sites. By “multiplex” is meant that two or more distinct foreign molecules may be delivered to the same target site or to different target sites (e.g., adjacent target sites, for instance adjacent target cells). The foreign molecules are different from each other, e.g., by different nucleic acid or amino acid sequence. In some instances the number of distinct foreign molecules is 2 or more, such as 4 or more, 6 or more, 8 or more, or 10 or more distinct foreign molecules. As such, in some cases, the biolistic barrels, devices and kits may include two or more distinct foreign molecules that each may be specifically delivered to different target sites, such as 2 or more, or 4 or more, 6 or more, 8 or more, or 10 or more distinct target sites. For example, the multiplex delivery of two distinct foreign molecules to two different target sites may include the multiplex transfection of two different nucleic acids or proteins into two different cells. Embodiments of the present disclosure may be used for the multiplex delivery of two or more distinct foreign molecules to one or more target sites either simultaneously or sequentially. For example, two or more distinct foreign molecules may be delivered to target sites simultaneously (e.g., at substantially the same time). In these embodiments, the two or more distinct foreign molecules may be provided in the same cartridge. In other embodiments, the biolistic barrels may be used to deliver two or more distinct foreign molecules sequentially. For example, a first foreign molecule may be delivered first in time, and then subsequently a second foreign molecule may be delivered to the same or a different target site. In these embodiments, the two or more distinct foreign molecules may be provided in different cartridges.

### Kits

Also provided are kits for use in practicing the methods. The kits at least include a biolistic barrel, e.g., as described above. In certain embodiments, the kits also include a needle or cannula, as described above, configured to attach to the distal end particle outlet of the biolistic barrel. In certain embodiments, the biolistic barrel and needle may be integrated into a biolistic device, such that they are components of the same device. In certain embodiments, the kits further include one or more biolistic particles, where the biolistic barrel, needle and biolistic particles may be present in separate containers in the kit. Alternatively, the kit may include at least one biolistic particle that is coated with an agent that can be delivered to a target site. Agents that can be delivered to a target site include, e.g., as described above, DNA, RNA, marker dyes, drugs and vaccines. As mentioned above, the kit components may be present in separate containers. Alternatively, the components may be present as a packaged element, such as those described above.

In addition to above-mentioned components, the kits may further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded.

The following examples are offered by way of illustration and not by way of limitation.

### EXAMPLES

In Vivo Transfection Experiments were Performed as Described Below in Experiments 1 and 2.

Experiment 1

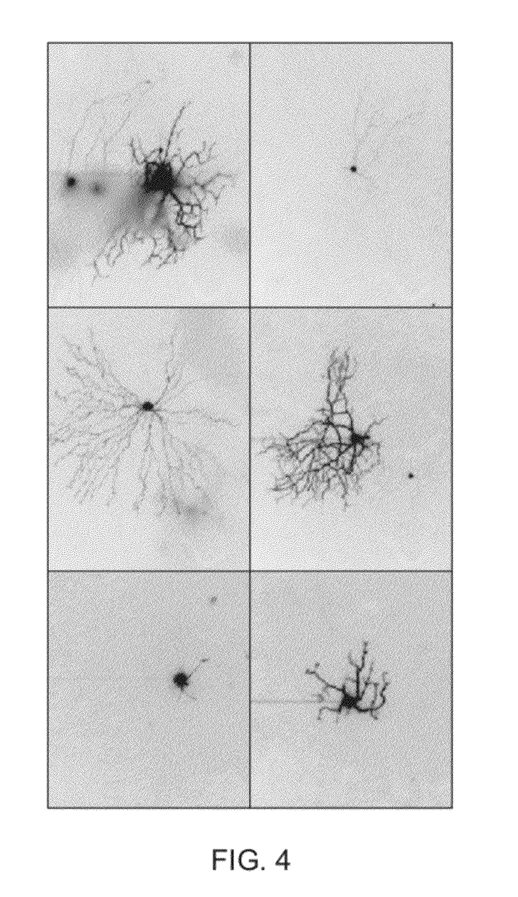

In vivo transfection of retinal neurons was performed as follows (results are shown in FIG. 4). Gold microcarriers (1.6 μm diameter) were complexed with DNA (25 μg) encoding the enhanced green fluorescent protein (eGFP) under transcriptional control of a hybrid CMV (cytomegalovirus) enhancer and chicken-beta actin promoter (referred to collectively as CAG promoter). The gold/DNA was precipitated onto the inner surface of ethylene tetrafluoroethylene (ETFE) tubing and loaded into the cartridge/barrel assembly. Positive pressure (˜1 psi) gas (95% O₂, 5% CO₂) was applied to the low pressure inlet. A 16 gauge needle was fitted to the barrel assembly. Under visualization through a stereoscopic operating microscope, the needle tip was advanced through the pars plana and vitreous humor to within 1 mm of the peripheral retina of the right eye of an anesthetized New Zealand White Rabbit. A small gas bubble was observed forming a clear fluid-free trajectory between the needle tip and the retina. A burst of helium gas (20 psi) regulated by three 0.174 psi blow-off valves was then manually triggered to propel gold/DNA into the retina. The needle was removed and the rabbit was observed post operatively until ambulatory. The animal was then euthanized 24 hours later and the retina was prepared for confocal microscopic imaging in a flat-mount preparation. eGFP in live (unfixed) retinal ganglion cells was visualized by 488 nm laser excitation and 510 nm emission using an Olympus FV1000/BX61 fixed-stage upright laser scanning confocal microscope equipped with argon and helium-neon lasers and a 20×/0.95NA water immersion objective lens.

FIG. 4 shows a magnified view of the rabbit retinal neurons 24 hours after biolistic transfection. As seen in FIG. 4, a variety of different morphology retinal neurons were observed to express green fluorescent protein (eGFP) following in vivo gene transfer of CAG-GFP plasmid to the rabbit retina.

Experiment 2

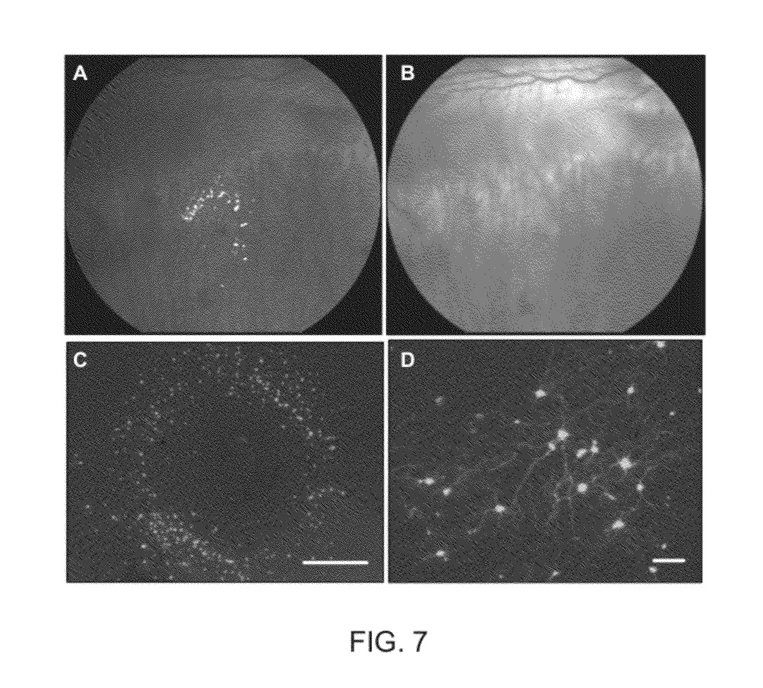

In vivo transfections were performed as follows (results are shown in FIG. 7). Biolistic particles were prepared by calcium phosphate-mediated precipitation of twenty-five (25) micrograms of plasmid DNA containing the tdTomato cDNA expressed by the Chicken beta-actin promoter onto the surface of twenty-five (25) milligrams of 1.6 micrometer diameter gold microparticles. Cartridges were prepared by depositing the DNA-complexed microparticles onto the inner surface of 0.125 inch (0.3175 cm) O.D. ethylene tetrafluoroethylene (ETFE) tubing, which was cut into 0.5 inch (1.27 cm) segments for loading into the biolistic device. Two-month old New Zealand White Rabbits were then anesthetized by subcutaneous injection of 2 ml Ketamine/Xylazine mixture (35 mg/kg and 5 mg/kg, respectively). Following anesthetization, eyes were dilated using 1 drop per eye of 2.5% phenylephrine hydrochloride and then 1% atropine sulfate. To anesthetize and immobilize the eye for the surgery, a drop of 0.5% proparacaine, a topical ophthalmic anesthetic, was applied. A shelving puncture was then made through the sclera with a sharp 16-gauge needle 1 mm posterior to the pars plana. A second blunt 18-gauge needle was then inserted into the puncture, which was attached to the biolistic device. The biolistic device included one pressure relief valve (cracking pressure of 0.6 psi). Constant and positive gas pressure (˜1 psi) (Bioblend, 95% O₂, 5% CO₂) was applied through the biolistic device to create a small gas bubble via the 18-gauge needle. An anterior chamber paracentesis was performed to enable the formation of the small gas bubble. The needle was advanced to approach the retina (within 2-4 mm) and a 20 millisecond burst of helium gas (20 psi) was applied to propel the DNA coated microparticles into the retina. The needle was withdrawn and the puncture closed by suture or cyanoacrylate based fast-acting adhesive, if needed. Two weeks following the in vivo transfection procedure, retinae were visualized in vivo via a RetCam II fluorescent fundus camera (Clarity Medical Systems, Inc., Pleasanton, Calif.) equipped with a 550 nm tdTomato excitation filter (see FIGS. 7A-7B). Rabbits were subsequently euthanized and retinae were dissected and visualized under low (see FIG. 7C) and high (FIG. 7D) magnification with a Zeiss Discovery V8 fluorescent stereo microscope (Carl Zeiss MicroImaging, LLC, New York) equipped with tdTomato excitation and emission (550 nm/580 nm) filters and captured via a Hamamatsu Orca CCD camera (Hamamatsu Photonics K.K., Japan). Transfected cells were observed to have the morphological features of ganglion, amacrine, Muller, and bipolar cells.

FIG. 7 shows images obtained two weeks after in vivo tdTomato transfection of rabbit retinal neurons with the biolistic device. FIGS. 7A and 7B show in vivo fundus fluorescent and bright field images, respectively, of rabbit retina two weeks following in vivo transfection with the biolistic device. FIGS. 7C and 7D show images of tdTomato expression shown ex vivo under low magnification in FIG. 7C and in high magnification in FIG. 7D. Scale bars are 500 μm in FIG. 7C and 50 μm in FIG. 7D.

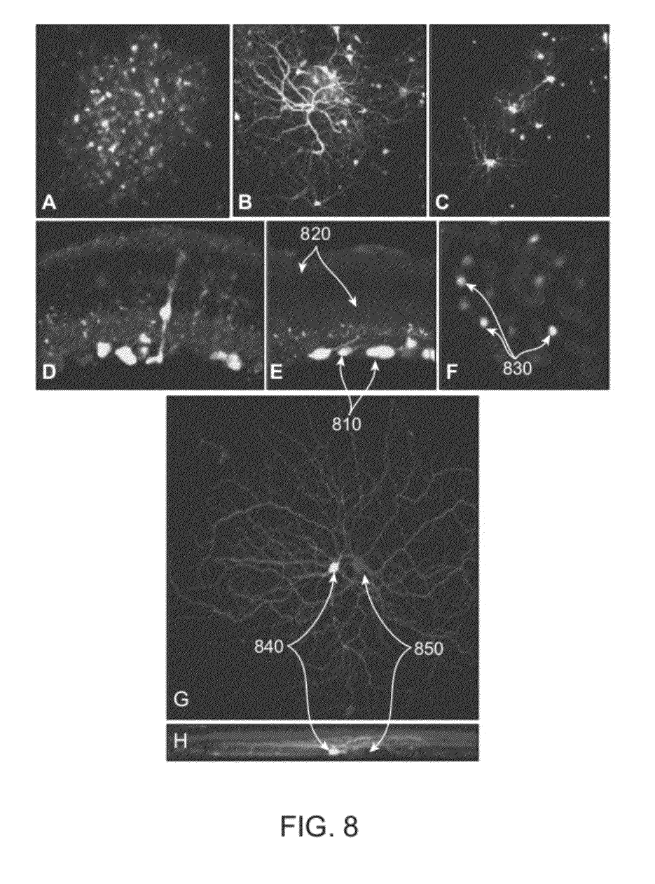

Multiplex rabbit retina transfections (results are shown in FIGS. 8G-8H) were performed in vivo as described above in Experiment 2. Biolistic particles were prepared by calcium phosphate-mediated precipitation of twenty-five (25) micrograms of plasmid DNA containing the tdTomato or eGFP cDNA expressed by the Chicken beta-actin or CAG promoter, respectively, onto the surface of twenty-five (25) milligrams of 1.6 micrometer diameter gold microparticles. In vivo transfections of rabbit retinae were performed as described above in Experiment 2 using the tdTomato and eGFP plasmid-containing microparticles. Retinae were visualized using either a RetCam II fluorescent fundus camera (Clarity Medical Systems, Inc., Pleasanton, Calif.) and/or a Zeiss Discovery V8 fluorescent stereo microscope (Carl Zeiss MicroImaging, LLC, New York) with a Hamamatsu Orca CCD camera (Hamamatsu Photonics K.K., Japan).

FIG. 8G (en face view) and FIG. 8H (transverse section) show fluorescent images of multiplex transfection of two plasmids with different promoters and transgenes (Cx36-eGFP (840) and HCN4-tdTomato (850)), which simultaneously labeled two different sets of rabbit ganglion cells.

Experiment 3

Ex Vivo Transfection Experiments were Performed as Described in Experiment 3, and as Shown in FIGS. 8A-8F.

Ex vivo transfections were performed as follows (results are shown in FIGS. 8A-8F). Biolistic particles were prepared by calcium phosphate-mediated precipitation of twenty-five (25) micrograms of plasmid DNA containing the tdTomato or eGFP cDNA expressed by the Chicken beta-actin or CAG promoter, respectively, onto the surface of twenty-five (25) milligrams of 1.6 micrometer diameter gold microparticles. Cartridges were prepared by depositing the DNA-complexed microparticles onto the inner surface of 0.125 inch (0.3175 cm) O.D. ethylene tetrafluoroethylene (ETFE) tubing, which was cut into 0.5 inch (1.27 cm) segments for loading into the biolistic device. Eyes were enucleated from a recently deceased Rhesus Macaque primate and ex vivo transfections were performed on whole intact globes as described above for in vivo rabbit transfections. High, medium, and low density transfections were performed by depositing 25, 10, or 5 milligrams, respectively, of the 1.6 μm gold/DNA mixture onto the inner surface of the ETFE tubing cartridge. Retinae were visualized using either a RetCam II fluorescent fundus camera (Clarity Medical Systems, Inc., Pleasanton, Calif.) or a Zeiss Discovery V8 fluorescent stereo microscope (Carl Zeiss MicroImaging, LLC, New York) with a Hamamatsu Orca CCD camera (Hamamatsu Photonics K.K., Japan).

Primate retina transfections (shown in FIGS. 8A-8F) were performed as described above in Experiment 3 in freshly enucleated intact eyes which were then cultured for 72 hr. FIGS. 8A-8F show fluorescent images of retinal neurons transfected with a biolistic device, according to embodiments of the present disclosure. FIGS. 8A, 8B and 8C show fluorescent images of primate retina (viewed en face) transfected with eGFP at different densities; high density transfection (FIG. 8A), medium density transfection (FIG. 8B), and low density transfection (FIG. 8C). FIG. 8D shows a fluorescent image of a transverse section of eGFP expression driven by CAG promoter in neurons and radial Muller glia. FIG. 8E shows a fluorescent image depicting cell specific hCx36 promoter driven eGFP expression in ganglion cells 810. 4′,6-diamidino-2-phenylindole (DAPI) labeled nuclei 820 appeared blue in the fluorescent image. FIG. 8F shows a fluorescent image of apoptotic cells 830 stained green with Yo-Pro following 72 hour culture of primate retina explant. The apoptotic cells 830 were only observed at the retinal edge and were not visible in regions transfected with tdTomato (not shown).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A biolistic barrel, the barrel comprising:
(a) a proximal end elevated pressure gas inlet;
(b) a distal end particle outlet; and
(c) at least one valve.

2. The biolistic barrel according to claim 1, wherein the barrel is configured so that a particle stream exiting the device through the distal end particle outlet has a positive pressure of 10 psi or less.

3. The biolistic barrel according to claim 1, wherein the at least one valve comprises a blow-off valve.

4. The biolistic barrel according to claim 3, wherein the barrel comprises three blow-off valves.

5. The biolistic barrel according to claim 1, wherein the distal end particle outlet is in fluid communication with an additional delivery device.

6. The biolistic barrel according to claim 5, wherein the additional delivery device is a needle, a catheter or a cannula.

7. The biolistic barrel according to claim 1, further comprising a cartridge holder.

8. The biolistic barrel according to claim 7, wherein the cartridge holder is configured to position one or more cartridges in fluid communication with the biolistic barrel.

9. The biolistic barrel according to claim 8, wherein the cartridge holder is configured to rotatably position two or more cartridges in fluid communication with the biolistic barrel.

10. The biolistic barrel according to claim 1, further comprising a low pressure gas inlet.

11. The biolistic barrel according to claim 10, wherein the low pressure gas inlet is positioned between the proximal end elevated pressure gas inlet and the distal end particle outlet.

12. A biolistic device comprising:
(a) a pressurized gas source coupler;
(b) a biolistic barrel, the barrel comprising:
(i) a proximal end elevated pressure gas inlet;
(ii) a distal end particle outlet; and
(iii) at least one valve;
(c) a cartridge holder; and
(d) an actuator for actuating the delivery device.

13. The biolistic device according to claim 12, further comprising a low pressure gas inlet.

14. The biolistic barrel according to claim 13, wherein the low pressure gas inlet comprises a valve.

15. The biolistic device according to claim 13, wherein the low pressure gas inlet is positioned between the proximal end elevated pressure gas inlet and the actuator.

16. The biolistic device according to claim 15, wherein the cartridge holder is positioned between the proximal end elevated pressure gas inlet and the distal end particle outlet.

17. The biolistic device according to claim 13, wherein the low pressure gas inlet is positioned between the proximal end elevated pressure gas inlet and the distal end particle outlet.

18-27. (canceled)

28. A method of delivering a molecule to a target site, the method comprising:
(a) positioning a biolistic delivery system in delivery relationship with the target site, wherein the system comprises:
(i) a pressurized gas source coupler;
(ii) a biolistic barrel, the barrel comprising:
a proximal end elevated pressure gas inlet;
a distal end particle outlet; and
at least one valve;
(iii) a biolistic particle loaded cartridge in a cartridge holder; and
(iv) an actuator for actuating the delivery device; and
(b) actuating the actuator to deliver the biolistic particle to the target site.

29-36. (canceled)

37. A kit for delivering a molecule to a target site, the kit comprising:
(a) a biolistic barrel, the barrel comprising:
(i) a proximal end elevated pressure gas inlet;
(ii) a distal end particle outlet; and
(iii) at least one valve; and
(b) a needle capable of being in fluid communication with the biolistic barrel.

38. The kit according to claim 37, wherein the kit further comprises at least one biolistic particle.
